(19) United States
(12) Patent Application Publication
(10) Pub. No.: US 2005/0008662 A1
(43) Pub. Date: Jan. 13, 2005

(54) Use of IV emulsions with different triglyceride composition, particle size and apolipoprotein E for targeted tissue delivery of hydrophobic compounds

(75) Inventors: Richard J. Deckelbaum, Hastings-on-Hudson, NY (US); Yvon A. Carpentier, Brussels (BE)

Correspondence Address: John P. White;Cooper & Dunham LLP, 1185 Avenue of the Americas, New York, NY, 10036, US

(73) Assignees: The Trustees of Columbia University in the City of New York; Universite libre de Bruxelles

(21) Appl. No.: 10/900,626

(22) Filed: Jul. 27, 2004

Related U.S. Application Data
(62) Division of application No. 10/033,629, filed on Dec. 28, 2001.
(60) Provisional application No. 60/258,654, filed on Dec. 29, 2000.

(51) Int. Cl. A61K 9/00; A61K 31/205; A61K 47/00

(52) U.S. Cl. 424/400; 514/786

(57) ABSTRACT

This invention provides compositions in the form of emulsions comprising pharmaceutical agents, triglycerides and emulsifiers. This invention also provides methods for delivering pharmaceutical agents to predetermined tissues comprising administering the instant compositions. This invention also provides methods of making the instant compositions.

This application claims the benefit of copending U.S. Provisional Application No. 60/258,654, filed Dec. 29, 2000, the contents of which are hereby incorporated by reference.

The invention disclosed herein was made with Government support under grant number HL40404 from the National Institutes of Health, U.S. Department of Health and Human Services. Accordingly, the U.S. Government has certain rights in this invention.

### BACKGROUND

Throughout this application, various references are referred to within parentheses. Disclosures of these publications in their entireties are hereby incorporated by reference into this application to more fully describe the state of the art to which this invention pertains. Full bibliographic citation for these references may be found at the end of this application, preceding the claims.

Many attempts have been made to increase the concentration of biologically active substances, e.g., an anti-tumor drug, in a certain organ or in certain target cells in order to increase the efficacy of a treatment and to reduce the side effects. One way to accomplish this maybe to link the drug to a carrier, e.g., a macromolecule. The rationale is that the macromolecule should have a high uptake by the target cell or that the linked carrier/drug in other aspects would give better efficacy than would be the case with the free drug. A number of macromolecules have been investigated with respect to their use as a carriers, such as DNA, liposomes, lipid microspheres, red blood ghost cells, lectines, different proteins such as antibodies, peptide hormones, glucoproteins and lipid amino acid conjugates.

Yamaguchi and Mizushima have described the use of lipid microspheres for drug delivery (Crit. Rev. Ther. Drug Carrier Syst. 11(4):215-29, 1994.). In brief, they have shown that lipid microspheres (with diameter of 0.2 microns) prepared from soybean oil and lecithin are promising carriers in vivo. The corticosteroids, nonsteroid anti-inflammatory drugs and prostaglandins, which were incorporated into these carrier particles, showed an increase in the drug potency. Yamaguchi and Mizushima also showed that the creation of a stable lipid microsphere drug delivery system is possible.

On the other hand our work (Treskova et al JPEN 23(5):253-259, 1999) showed that lipid emulsions with different structure have different properties in terms of blood clearance. We showed that addition of fish oil (Ω-3) triglycerides to medium/long chain (MCT/LCT) containing emulsions did not decrease, and in fact even enhanced the ability of lipoprotein lipase to release triglyceride fatty acids from these particles. Inclusion of MCT into emulsion particles markedly decreases the hydrolysis of both LCT and ω-3 within the emulsions particle, an effect likely associated with displacement of LCT and Ω-3 from the emulsions surface by MCT. In MCT containing emulsions, and in the poorly hydrolyzed pure Ω-3 triglyceride emulsion, most long chain fatty acids are delivered to tissues in emulsion triglycerides (as triglyceride remnant particles) rather than as free fatty acids.

An effective means of tissue-targeted delivery of biologically active substances such as pharmaceutical agents is still sought. The invention disclosed here provides such a means.

### SUMMARY

This invention provides a composition in the form of an emulsion comprising:
(a) a therapeutically effective amount of a pharmaceutical agent;
(b) an amount of a fish oil predetermined so as to deliver the pharmaceutical agent to a predefined tissue in a subject; and
(c) an amount of an emulsifier sufficient to result in the composition forming the emulsion.

This invention further comprises the instant composition, wherein the fish oil is an Ω-3 triglyceride.

This invention further provides the instant composition, wherein the predefined tissue is an extrahepatic tissue and the Ω-3 triglyceride preferentially effects delivery of the pharmaceutical agent to the extrahepatic tissue.

This invention also provides a method of making the instant composition comprising:
(i) admixing (a) a therapeutically effective amount of a pharmaceutical agent, (b) an amount of a fish oil predetermined so as to deliver the pharmaceutical agent to a predefined tissue in a subject, and (c) an amount of an emulsifier sufficient to result in the composition forming the emulsion,
(ii) and treating the resulting admixture so as to form an emulsion.

This invention also provides a composition in the form of an emulsion comprising:
(a) a therapeutically effective amount of a pharmaceutical agent;
(b) an amount of a medium chain triglyceride;
(c) an amount of a long-chain triglyceride; and
(d) an amount of an emulsifier sufficient to result in the composition forming the emulsion;
wherein the amount of the medium chain triglyceride relative to the amount of the long-chain triglyceride are predetermined so as to deliver the pharmaceutical agent to a predefined tissue in a subject.

This invention further provides the instant composition, wherein the amount of the medium chain triglyceride relative to the amount of the long-chain triglyceride is in a ratio of about one to one by weight.

This invention also provides a method of making the instant composition comprising:
(i) admixing (a) a therapeutically effective amount of a pharmaceutical agent, (b) an amount of a medium chain triglyceride, (c) an amount of a long-chain triglyceride, and (d) an amount of an emulsifier sufficient to result in the composition forming the emulsion, wherein the amount of the medium chain triglyceride relative to the amount of the long-chain triglyceride are predetermined so as to deliver the pharmaceutical agent to a predefined tissue in a subject;
(ii) and treating the resulting admixture so as to form an emulsion.

This invention also provides a composition in the form of an emulsion comprising:
(a) a therapeutically effective amount of a pharmaceutical agent;
(b) an amount of a fish oil;
(c) an amount of a medium chain triglyceride;
(d) an amount of a long-chain triglyceride; and
(e) an amount of an emulsifier sufficient to result in the composition forming an emulsion;
wherein each of the amount of fish oil, the amount of medium chain triglyceride and the amount of long-chain triglyceride is predetermined so as to deliver the pharmaceutical agent to a predefined tissue in a subject.

This invention further provides the instant composition, wherein the amount of the medium chain triglyceride relative to the amount of the long-chain triglyceride relative to the amount of the fish oil is in a ratio of about 5:4:1 by weight.

This invention further provides the instant composition, wherein the fish oil is an Ω-3 triglyceride.

This invention further provides the instant composition, wherein the predefined tissue is an extrahepatic tissue and the Ω-3 triglyceride preferentially effects delivery of the pharmaceutical agent to the extrahepatic tissue.

This invention also provides a method of making the instant composition comprising:
(i) admixing (a) a therapeutically effective amount of a pharmaceutical agent, (b) an amount of a fish oil, (c) an amount of a medium chain triglyceride, (d) an amount of a long-chain triglyceride, and (e) an amount of an emulsifier sufficient to result in the composition forming an emulsion, wherein each of the amount of fish oil, the amount of medium chain triglyceride and the amount of long-chain triglyceride is predetermined so as to deliver the pharmaceutical agent to a predefined tissue in a subject;
(ii) and treating the resulting admixture so as to form an emulsion.

This invention further provides the instant compositions, wherein more than 80% of the particles in the emulsion have a diameter between 30 and 150 nm.

This invention also provides a method of delivering a pharmaceutical agent to an hepatic tissue in a subject which comprises administering to the subject the instant composition.

This invention further provides the instant compositions, wherein more than 80% of the particles in the emulsion have a diameter between 150 and 350 nm.

This invention also provides a method of delivering a pharmaceutical agent to an extrahepatic tissue in a subject which comprises administering to the subject the instant composition.

This invention also provides a method of delivering a pharmaceutical agent to a predefined tissue in a subject comprising administering to the subject the composition of any of instant compositions, so as to preferentially deliver the pharmaceutical agent to the predefined tissue in the subject.

This invention also provides a composition in the form of an emulsion comprising:
(a) a therapeutically effective amount of a pharmaceutical agent;
(b) an amount of a triglyceride;
(c) an amount of an emulsifier sufficient to result in the composition forming the emulsion; and
(d) an amount of a ligand which specifically binds to a predefined tissue;
wherein the amount of the triglyceride is predetermined to deliver the pharmaceutical agent to the predefined tissue, and the amount of ligand preferentially effects the delivery of the pharmaceutical agent to the predefined tissue.

This invention further provides the instant composition, wherein the ligand is an apolipoprotein E.

This invention further provides the instant composition, wherein the apolipoprotein E is human apolipoprotein E or a homolog thereof differing by fewer than 3 amino acids, but having the biological activity of naturally occurring human apolipoprotein E.

This invention also provides a method for delivering a pharmaceutical agent to a tissue in a subject expressing on its surface a low density lipoprotein receptor, a low density lipoprotein-related protein receptor, a very low density lipoprotein receptor or a proteoglycan comprising administering to the subject the instant composition, so as to preferentially deliver the pharmaceutical agent to the tissue in the subject.

This invention further provides the instant method, wherein the tissue is a hepatic tissue.

This invention further provides the instant method, wherein the tissue is a reticulo-endothelial tissue.

This invention also provides a method of making the instant composition comprising:
(i) admixing (a) a therapeutically effective amount of a pharmaceutical agent, (b) an amount of a triglyceride, (c) an amount of an emulsifier sufficient to result in the composition forming the emulsion, and (d) an amount of a ligand which specifically binds to a predefined tissue, wherein the amount of the triglyceride is predetermined to deliver the pharmaceutical agent to the predefined tissue, and the amount of ligand preferentially effects the delivery of the pharmaceutical agent to the predefined tissue,
(ii) and treating the resulting admixture so as to form an emulsion.

This invention further provides the instant methods, wherein the administration comprises intravenous injection.

This invention further provides the instant methods, wherein the subject is a mammal.

This invention further provides the instant method, wherein the mammal is a human being.

This invention also provides a composition in the form of an emulsion comprising:
(a) a therapeutically effective amount of a pharmaceutical agent;
(b) an amount a triglyceride;
(c) an amount of an emulsifier sufficient to result in the composition forming the emulsion;
wherein the amount of the triglyceride is predetermined so as to deliver the pharmaceutical agent to a predefined tissue in a subject.

This invention further provides the instant composition, wherein the triglyceride comprises a medium-chain triglyceride or a long-chain triglyceride.

This invention also provides a method of making the instant composition comprising:
(i) admixing (a) a therapeutically effective amount of a pharmaceutical agent, (b) an amount a triglyceride, (c) an amount of an emulsifier sufficient to result in the composition forming the emulsion, wherein the amount of the triglyceride is predetermined so as to deliver the pharmaceutical agent to a predefined tissue in a subject;
(ii) and treating the resulting admixture so as to form an emulsion.

### BRIEF DESCRIPTION OF THE FIGURES

FIG. 1. This figure shows the differences between hepatic uptake of the different emulsions. The liver uptake of LCT, MCT/LCT and Ω-3 triglyceride was similar for LCT, MCT/LCT, and Ω-3 emulsions (39%±3.9%, 46%±3.6% and 34%±3.2%) of recovered ³H-CE, respectively. However, blending 10% (by weight) of Ω-3 triglyceride with MCT/LCT to produce MCT/LCT/Ω-3, decreased liver uptake to 23%±2.2%.

FIG. 2. This figure shows the lung uptake of ³H-CE for pure Ω-3 triglyceride (FO) was 7.5 times higher than that of LCT (900×10³±20×10³ DPM/gm vs. 120×10³±30×10³ DPM/gm, p=0.001).

FIG. 3. This figure shows the brain uptake of pure Ω-3 triglyceride was 2-3 times more than for other emulsions.

FIG. 4. This figure shows the blood clearance of IDL and VLDL (Emulsion-S) vs. chylomicron size particles (Emulsion-L) Clearance for the chylomicron type particles (1.2±0.3 pools/hr, 15±3.8 pools/hr, p<0.0001) is 10 times faster.

FIG. 5. This figure shows that percent-wise Emulsion-S had significantly higher liver uptake than that of Emulsion-L (71%±3.1%, vs. 28%±4.3%, p<0.0001).

FIG. 6. This figure shows there was an increase in lung uptake of the apolipoprotein E containing vs. apolipoprotein E negative emulsion (10×10³±1×10³ DPM/gm vs. 4.6×10³±0.3×10³ DPM/gm).

FIG. 7. This figure shows that LCT emulsion, containing apolipoprotein E had higher liver uptake than the apolipoprotein E negative emulsion (39%±6%, vs. 15%±2%, p=0.01).

FIG. 8. This figure shows Emulsion-L uptake vs. Emulsion-S was significantly higher in lung.

FIG. 9. This figure shows the higher blood clearance of LCT emulsion in the presence of Apolipoprotein E.

### DETAILED DESCRIPTION OF THE INVENTION

The following definitions are presented as an aid in understanding this invention:

| | |
|---|---|
| Apo E | Apoliporotein E |
| Ω-3 | Omega-3; |
| ³H-CE | ³H-cholesteryl oleoyl ether; |
| DNA | Deoxyribonucleic Acid; |
| DPM | Disintegrations per minute; |
| E. Coli | Escherichia Coli; |
| IDL | Intermediate Density Lipoprotein; |
| LCT | Long Chain Triglycerides; |
| MCT | Medium Chain Triglycerides; |
| nm | nanometers; and |
| VLDL | Very Low Density Lipoprotein. |

“Fish oil” includes synthetic fish oil, i.e. a fish oil that has been esterified or re-esterified.

A medium-chain triglyceride is a triglyceride composed of more than 90% fatty acids of C6 to C10 in length.

A long-chain triglyceride is a triglyceride composed of more than 90% fatty acids of C12 to C24 in length.

This invention provides a composition in the form of an emulsion comprising:
(a) a therapeutically effective amount of a pharmaceutical agent;
(b) an amount of a fish oil predetermined so as to deliver the pharmaceutical agent to a predefined tissue in a subject; and
(c) an amount of an emulsifier sufficient to result in the composition forming the emulsion.

In one embodiment the fish oil comprises an Ω-3 triglyceride. In further embodiments the Ω-3 triglyceride comprises eicosapentaenoic acid and/or docosahexaenoic acid. In another embodiment the fish oil comprises at least 40% eicosapentaenoic acid and docosahexaenoic acid. In one embodiment the fish oil is a synthetic fish oil. In one embodiment the fish oil is a tridocohexanoin. In another embodiment the Ω-3 triglyceride comprises fatty acids of the following composition C12:0 0.4%; C14:0 6.2%; C16:0 12.6%; C18:0 1.3%; C18:1n9 6.8%; C18:2n6 1.4%; C18:3n6 0.2%; C18:3n3 1.3%; C20:1 1.4%; C18:4n3 4.7%; C20:4n6 2.6%; C20:5n3 34.4%; C22:4n6 1.8%; C22:5n3 4.1%; C22:6n3 20.7%, wherein C followed by a number represents the length of the carbon backbone and wherein n followed by a number refers to the placement of double bonds. In one embodiment the composition in the form of an emulsion comprises a total of between 9 and 21 g of triglyceride per 100 ml emulsion. In a preferred embodiment the composition in the form of an emulsion comprises a total of 20 g of triglyceride per 100 ml emulsion. In an alternative embodiment the emulsion comprises a total of 10 g of triglyceride per 100 ml emulsion.

In one embodiment the emulsifier is a surfactant. In a further embodiment the surfactant is a phospholipid. Examples of phospholipids are egg yolk lecithin, a biologic phospholipid, a phosphatidylcholine with fixed fatty acyl chain composition, a glycophospholipid or a phosphatidylethanolamine. In one embodiment the emulsifier is 1.2 mg of egg yolk lecithin/100 ml emulsion.

This invention further provides the instant composition, wherein the predefined tissue is an extrahepatic tissue and the Ω-3 triglyceride preferentially effects delivery of the pharmaceutical agent to the extrahepatic tissue. In one embodiment the extrahepatic tissue is a neural tissue. In a further embodiment the neural tissue is brain tissue. In another embodiment the extrahepatic tissue is lung. In other embodiments the extrahepatic tissue is cardiac tissue, spleen, adipose tissue or muscle. Other examples of extrahepatic tissue include adrenal and kidney tissues.

This invention also provides a method of making the instant composition comprising:
(i) admixing (a) a therapeutically effective amount of a pharmaceutical agent, (b) an amount of a fish oil predetermined so as to deliver the pharmaceutical agent to a predefined tissue in a subject, and (c) an amount of an emulsifier sufficient to result in the composition forming the emulsion,
(ii) and treating the resulting admixture so as to form an emulsion.

Emulsions are made by standard methods, for example emulsifying using the egg yolk lecithin, 1.2 g/100 ml and prepared so as to contained 20 g Triglyceride/100 ml emulsion.

This invention also provides a composition in the form of an emulsion comprising:
(a) a therapeutically effective amount of a pharmaceutical agent;
(b) an amount of a medium chain triglyceride;
(c) an amount of a long-chain triglyceride; and
(d) an amount of an emulsifier sufficient to result in the composition forming the emulsion;
wherein the amount of the medium chain triglyceride relative to the amount of the long-chain triglyceride are predetermined so as to deliver the pharmaceutical agent to a predefined tissue in a subject.

This invention further provides the instant composition, wherein the amount of the medium chain triglyceride relative to the amount of the long-chain triglyceride is in a ratio of about one to one by weight. Medium-chain triglycerides are triglycerides composed of more than 90% fatty acids of C6 to C10 in length. Long-chain triglycerides are triglycerides composed of more than 90% fatty acids of C12 to C24 in length. In one embodiment the LCT is derived from Soy Oil. In one embodiment the LCT is a triolein. In one embodiment the MCT is derived from Coconut Oil. In one embodiment the MCT is a trioctanoin. In one embodiment the MCT/LCT emulsion comprises fatty acids of the following composition—C8:0 31.41%; C10:0 17.5%; C12:0 0.29%; C14:0 0.01%; C16:0 5.1%; C16:1 0.05%; C18:0 2.24%; C18:1 12.08%; C18:2(n-6) 27.46%; C18:3(n-3) 2.9%; C20:0 0.75%; C20:4(n-6) 0.19% wherein C followed by a number represents the length of the carbon backbone and wherein n followed by a number refers to the placement of double bonds.

This invention also provides a method of making the instant composition comprising:
(i) admixing (a) a therapeutically effective amount of a pharmaceutical agent, (b) an amount of a medium chain triglyceride, (c) an amount of a long-chain triglyceride, and (d) an amount of an emulsifier sufficient to result in the composition forming the emulsion, wherein the amount of the medium chain triglyceride relative to the amount of the long-chain triglyceride are predetermined so as to deliver the pharmaceutical agent to a predefined tissue in a subject;
(ii) and treating the resulting admixture so as to form an emulsion.

Emulsions are made by standard methods, for example emulsifying using the egg yolk lecithin, 1.2 g/100 ml and prepared so as to contained 20 g Triglyceride/100 ml emulsion. The weight ratio of LCT and MCT in the different emulsions are varied according to choice.

This invention also provides a composition in the form of an emulsion comprising:
(a) a therapeutically effective amount of a pharmaceutical agent;
(b) an amount of a fish oil;
(c) an amount of a medium chain triglyceride;
(d) an amount of a long-chain triglyceride; and
(e) an amount of an emulsifier sufficient to result in the composition forming an emulsion;
wherein each of the amount of fish oil, the amount of medium chain triglyceride and the amount of long-chain triglyceride is predetermined so as to deliver the pharmaceutical agent to a predefined tissue in a subject.

This invention further provides the instant composition, wherein the amount of the medium chain triglyceride relative to the amount of the long-chain triglyceride relative to the amount of the fish oil is in a ratio of about 5:4:1 by weight.

This invention further provides the instant composition, wherein the fish oil comprises an Ω-3 triglyceride. In further embodiments the Ω-3 triglyceride comprises eicosapentaenoic acid and/or docosahexaenoic acid. In another embodiment the fish oil comprises at least 40% eicosapentaenoic acid and docosahexaenoic acid. In one embodiment the fish oil is a synthetic fish oil. In one embodiment the fish oil is a tridocohexanoin. In one embodiment the LCT is derived from Soy Oil. In one embodiment the LCT is a triolein. In one embodiment the MCT is derived from Coconut Oil. In one embodiment the MCT is a trioctanoin

This invention further provides the instant composition, wherein the predefined tissue is an extrahepatic tissue and the Ω-3 triglyceride preferentially effects delivery of the pharmaceutical agent to the extrahepatic tissue. In one embodiment the extrahepatic tissue is a neural tissue. In a further embodiment the neural tissue is brain tissue. In another embodiment the extrahepatic tissue is lung. In other embodiments the extrahepatic tissue is cardiac tissue, spleen, adipose tissue or muscle. Other examples of extrahepatic tissue include adrenal and kidney tissues.

This invention also provides a method of making the instant composition comprising:
(i) admixing (a) a therapeutically effective amount of a pharmaceutical agent, (b) an amount of a fish oil, (c) an amount of a medium chain triglyceride, (d) an amount of a long-chain triglyceride, and (e) an amount of an emulsifier sufficient to result in the composition forming an emulsion, wherein each of the amount of fish oil, the amount of medium chain triglyceride and the amount of long-chain triglyceride is predetermined so as to deliver the pharmaceutical agent to a predefined tissue in a subject;
(ii) and treating the resulting admixture so as to form an emulsion.

Emulsions are made by standard methods, for example emulsifying using the egg yolk lecithin, 1.2 g/100 ml and prepared so as to contained 20 g Triglyceride/100 ml emulsion. The weight ratio of LCT/MCT/Ω-3 in the different emulsions are varied according to choice.

This invention further provides the instant compositions, wherein more than 80% of the particles in the emulsion have a diameter between 30 and 150 nm. To produce such

This invention also provides a method of delivering a pharmaceutical agent to an hepatic tissue in a subject which comprises administering to the subject the instant composition.

This invention further provides the instant compositions, wherein more than 80% of the particles in the emulsion have a diameter between 150 and 350 nm.

This invention also provides a method of delivering a pharmaceutical agent to an extrahepatic tissue in a subject which comprises administering to the subject the instant composition. In one embodiment the extrahepatic tissue is a neural tissue. In a further embodiment the neural tissue is brain tissue. In another embodiment the extrahepatic tissue is lung. In other embodiments the extrahepatic tissue is cardiac tissue, spleen, adipose tissue or muscle. Other examples of extrahepatic tissue include adrenal and kidney tissues

This invention also provides a method of delivering a pharmaceutical agent to a predefined tissue in a subject comprising administering to the subject the composition of any of instant compositions, so as to preferentially deliver the pharmaceutical agent to the predefined tissue in the subject. Examples of such tissue are hepatic and extrahepatic tissues. In one embodiment the extrahepatic tissue is a neural tissue. In a further embodiment the neural tissue is brain tissue. In another embodiment the extrahepatic tissue is lung. In other embodiments the extrahepatic tissue is cardiac tissue, spleen, adipose tissue or muscle. Other examples of extrahepatic tissue include adrenal and kidney tissues.

The delivery of an effective amount of a pharmaceutical agent effects treatment of a disease in the tissue wherein the pharmaceutical agent treats the disease and is present in an amount effective to do so. Such disease include tumors, hepatic disease, inflammation and diseases of extrahepatic tissues. Examples of pharmaceutical agents are anti-tumor drugs, immunosuppressives, anti-viral agents, hydrophobic compounds, a compound which is not water soluble, a leptin, a fluorescent tracer, a radioactive tracer, or vitamin E. Determining the effective amount of the instant pharmaceutical composition can be done based on animal data using routine computational methods.

This invention also provides a composition in the form of an emulsion comprising:
(a) a therapeutically effective amount of a pharmaceutical agent;
(b) an amount of a triglyceride;
(c) an amount of an emulsifier sufficient to result in the composition forming the emulsion; and
(d) an amount of a ligand which specifically binds to a predefined tissue;
wherein the amount of the triglyceride is predetermined to deliver the pharmaceutical agent to the predefined tissue, and the amount of ligand preferentially effects the delivery of the pharmaceutical agent to the predefined tissue.

This invention further provides the instant composition, wherein the ligand is an apolipoprotein E. This invention further provides the instant composition, wherein the apolipoprotein E is human apolipoprotein E or a homolog thereof differing by fewer than 3 amino acids, but having the biological activity of naturally occurring human apolipoprotein E. This invention also provides a method for delivering a pharmaceutical agent to a tissue in a subject expressing on its surface a low density lipoprotein receptor, a low density lipoprotein-related protein receptor, a very low density lipoprotein receptor or a proteoglycan comprising administering to the subject the instant composition, so as to preferentially deliver the pharmaceutical agent to the tissue in the subject. In one embodiment the tissue is a hepatic tissue. In another embodiment the tissue is a reticulo-endothelial tissue. In another embodiment the tissue is lung tissue.

This invention also provides a method of making the instant composition comprising:
(i) admixing (a) a therapeutically effective amount of a pharmaceutical agent, (b) an amount of a triglyceride, (c) an amount of an emulsifier sufficient to result in the composition forming the emulsion, and (d) an amount of a ligand which specifically binds to a predefined tissue, wherein the amount of the triglyceride is predetermined to deliver the pharmaceutical agent to the predefined tissue, and the amount of ligand preferentially effects the delivery of the pharmaceutical agent to the predefined tissue,
(ii) and treating the resulting admixture so as to form an emulsion.

Emulsions are made by standard methods, for example emulsifying using the egg yolk lecithin, 1.2 g/100 ml and prepared so as to contained 20 g Triglyceride/100 ml emulsion. Triglycerides include LCT, MCT and Ω-3 triglycerides. In the case of more than one triglyceride the weight ratio of triglycerides in the different emulsions are varied according to choice.

This invention further provides the instant methods, wherein the administration comprises intravenous injection.

This invention further provides the instant methods, wherein the subject is a mammal. In one embodiment the mammal is a human being.

This invention also provides a composition in the form of an emulsion comprising:
(a) a therapeutically effective amount of a pharmaceutical agent;
(b) an amount a triglyceride;
(c) an amount of an emulsifier sufficient to result in the composition forming the emulsion;
wherein the amount of the triglyceride is predetermined so as to deliver the pharmaceutical agent to a predefined tissue in a subject.

This invention further provides the instant composition, wherein the triglyceride comprises a medium-chain triglyceride or a long-chain triglyceride. In one embodiment the LCT is derived from Soy Oil. In one embodiment the LCT is a triolein.

In one embodiment the MCT is derived from Coconut Oil.

This invention also provides a method of making the instant composition comprising:
(i) admixing (a) a therapeutically effective amount of a pharmaceutical agent, (b) an amount a triglyceride, (c) an amount of an emulsifier sufficient to result in the composition forming the emulsion, wherein the amount of the triglyceride is predetermined so as to deliver the pharmaceutical agent to a predefined tissue in a subject;
(ii) and treating the resulting admixture so as to form an emulsion.

Emulsions are made by standard methods, for example emulsifying using the egg yolk lecithin, 1.2 g/100 ml and prepared so as to contained 20 g Triglyceride/100 ml emulsion.

Experiments

We synthesized various emulsions to target delivery of agents to tissues.

### EXAMPLE 1

The lipid emulsions were prepared by B. Braun GmbH (Melsungen, Germany) using standard industry methods for production of therapeutic emulsion in water. All emulsions were emulsified by the same egg yolk lecithin, 1.2 g/100 ml and contained 20 g Triglyceride/100 ml. The relative triglyceride composition (by weight) of the emulsions used for these experiments: a) LCT (100% soy oil); b) MCT/LCT (1/1, w/w); c) MCT/LCT/ω-3 (5:4:1, w/w) and d) Ω-3 (100% fish oil). The fatty acid composition of each emulsion was as follows: a) LCT—C14:0 0.01%; C16:0 10.07%; C16:1 0.09%; C18:0 4.25%; C18:1 23.8%; C18:2(n-6) 53.91%; C18:3(n-3), 5.78%; C20:0 1.74%; C20:4(n-6) 0.36%; b) MCT/LCT—C8:0 31.41%; C10:0 17.5%; C12:0 0.29%; C14:0 0.01%; C16:0 5.1%; C16:1 0.05%; C18:0 2.24%; C18:1 12.08%; C18:2(n-6) 27.46%; C18:3(n-3) 2.9%; C20:0 0.75%; C20:4(n-6) 0.19%; c) MCT/LCT/Ω-3—C8:0 31.2%; C10:0 20.1%; C16:0 5.8%; C18:0 2.3%; C:18:1 8.3%; C18:2(n-6) 22.9%; C18:3(n-3) 4.1%; C22:0 1.4%; C20:5(n-3) 2.3%; C22:6n3 1.7% and d) Ω-3—C12:0 0.4%; C14:0 6.2%; C16:0 12.6%; C18:0 1.3%; C18:1n9 6.8%; C18:2n6 1.4%; C18:3n6 0.2%; C18:3n3 1.3%; C20:1 1.4%; C18:4n3 4.7%; C20:4n6 2.6%; C20:5n3 34.4%; C22:4n6 1.8%; C22:5n3 4.1%; C22:6n3 20.7%, wherein n followed by a number refers to the placement of double bonds. Emulsion particle size was measured by the manufacturer and all emulsions had similar diameters (˜300nm) with no significant differences between them. ³H-cholesteryl oleoyl ether (³H-CE) was obtained from Amersham/Pharmacia Biotech, UK, Ltd and was used as a marker of triglyceride remnant particle and as a model of biologically active hydrophobic substance. In order to generate radiolabeled emulsions, containing ³H-CE, 0.001 Ci/200 mg triglyceride was added to a small amber glass vial, and the solvent was slowly evaporated to dryness under N₂. Immediately upon reaching dryness, 150 μL of the emulsion was added to the vial. The vial was mixed vigorously and allowed to sit on the batch for 30 min. Following the same procedure, another two portions of emulsion were added to a total of 500 μL emulsion volume. The emulsion was sonicated 3 times on ice for 20 sec each at power setting of 40 Watt using Branson Sonifier Cell Disruptor (Model W185, Branson Scientific, Inc., Plainview, N.Y.) to incorporate the ³H-CE into the emulsion particle. The resulting emulsion was stored in the dark, at 40° C. for up to 5 days prior to use in experiments. Elution profiles of labeled emulsions on Sepharose CL2B column showed that all ³H-CE co-eluted with the emulsion particles. Thus, all radiolabel was in the emulsion.

To assess whether the ³H-CE had been incorporated in a similar manner and to a similar extent into each emulsion and to demonstrate that the sonication had not disrupted the emulsion particles, analyzes of the emulsion was done after sonication. Small aliquots of sonicated and unsonicated emulsion were transferred into 75 μL capillary tubes and centrifuged for 20 min in a hematocrit centrifuge. After centrifugation, the tubes were cut into 6 sections (0.13 cm in length) and triglyceride and phospholipid concentrations were assayed in corresponding sections of sonicated and unsonicated emulsions. For sonicated emulsion the radioactivity present in each section was measured. All emulsion, whether sonicated or not had the same Triglyceride/phospholipid ratios in the corresponding sections of the tube. As well, >90% of radiolabel was in the Triglyceride-rich emulsion fraction of tube. Prior to injection, emulsion equal to 2 mg Triglyceride/100 g body weight per animal was aspirated into a 100 μL syringe and diluted with 0.9% NaCl to a total volume of 50 μL.

The blood clearance of 4 different emulsions was studied. Extrapolation of the rapid clearance phase for each emulsion back to time 0 gave an estimation of the initial amount of emulsion in blood. There were no significant differences for recoveries between three different emulsions: i.e. LCT, MCT:LCT, and MCT:LCT:Ω-3. However, pure fish oil had a significantly higher clearance. For example compared to LCT, the calculated fraction clearance coefficient (FCR) for Ω-3 emulsion was 21.4±3.8 vs. 17.0±3.2 pools/h and 22.4±2.4 vs. 15.9±1.3 pools/h in fed and fasted states respectively, p<0/01.

The fractional clearance of LCT, MCT/LCT, MCT/LCT/Ω-3 emulsions were similar (18.9±0.6 pools/hr, 17.0±0.96 pools/hr and 16.5±1.08 pools/hr).

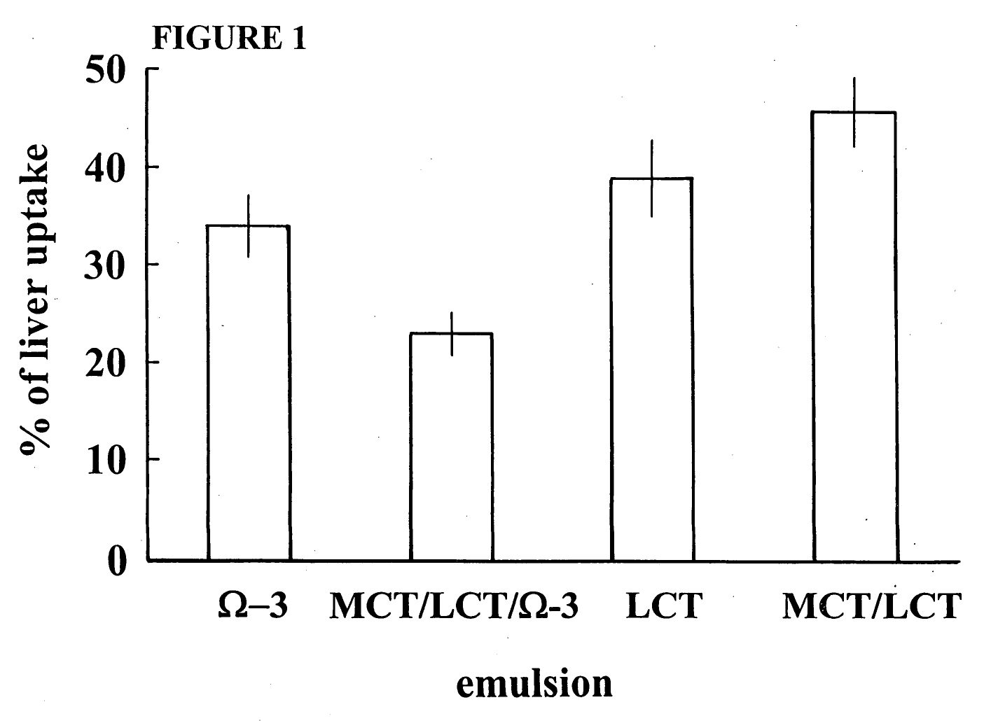

We next assessed potential differences between hepatic vs. extrahepatic tissue uptake of the different emulsions (FIG. 1). The liver uptake of LCT, MCT/LCT and Ω-3 triglyceride was similar for LCT, MCT/LCT, and Ω-3 emulsions (39%±3.9%, 46%±3.6% and 34%±3.2%) of recovered ³H-CE, respectively. However, blending 10% (by weight) of Ω-3 triglyceride with MCT/LCT to produce MCT/LCT/Ω-3, decreased liver uptake to 23%±2.2%. This was significantly less than LCT/MCT (46%±3.6%, p<0.0001) and LCT (39%±3.9%, p=0.002) suggesting that the addition of Ω-3 triglyceride to MCT/LCT increases its distribution to extrahepatic tissues.

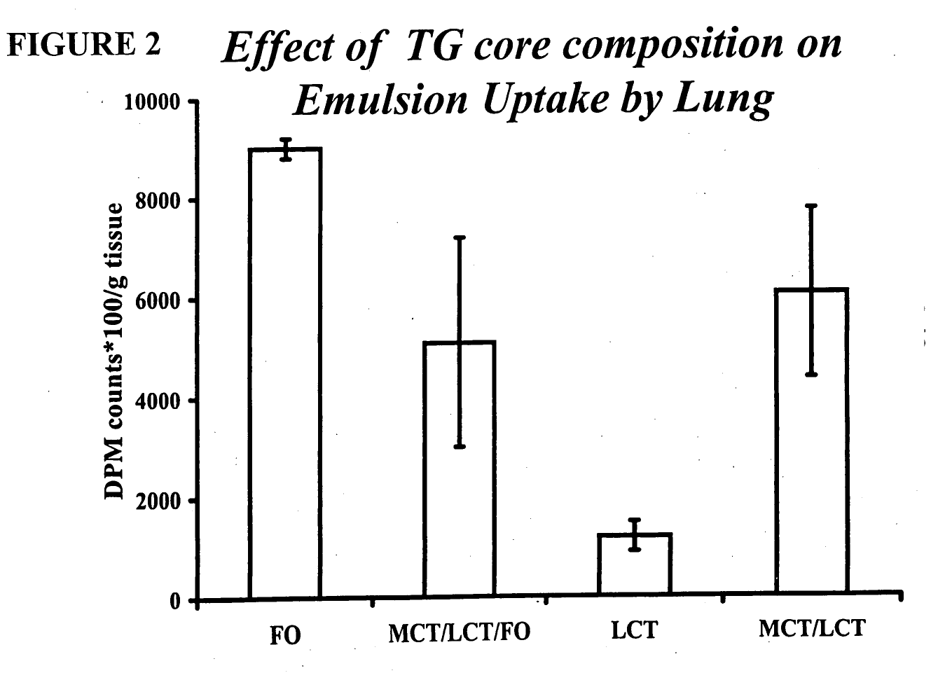

The lung uptake of ³H-CE for pure Ω-3 triglyceride was 7.5 times higher than that of LCT (90×10³±20×10³ DPM/gm vs. 120×10³±30×10³ DPM/gm, p=0.001) (FIG. 2).

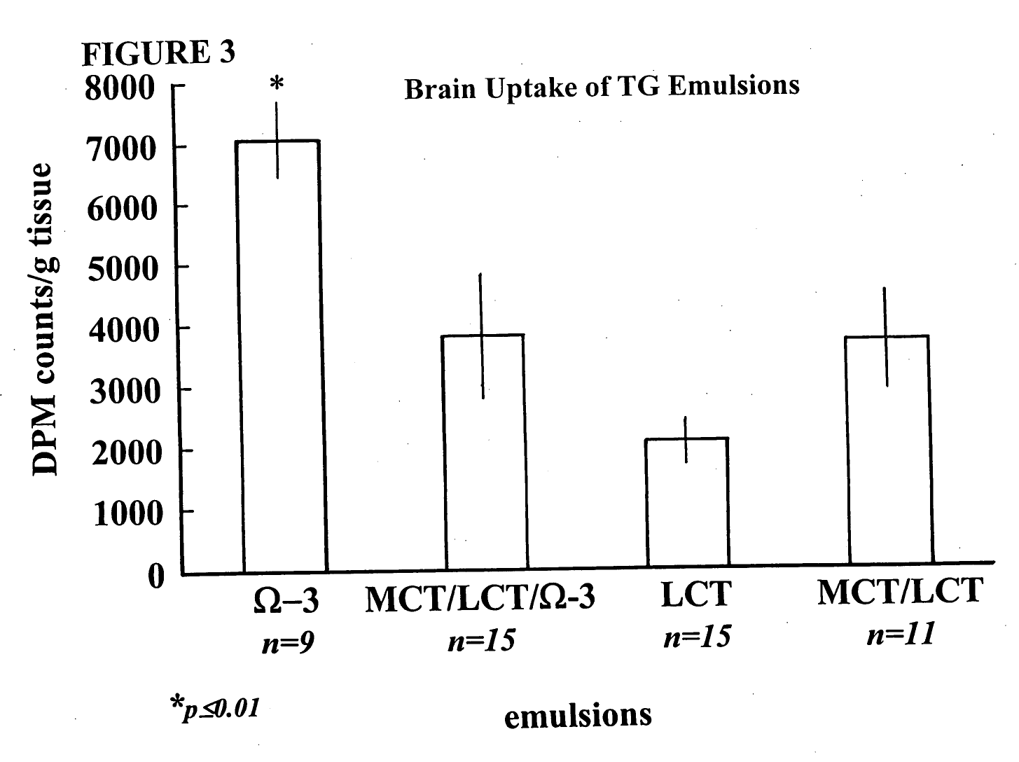

The brain uptake of pure Ω-3 triglyceride was 2-3 times more than for other emulsions) (FIG. 3).

### EXAMPLE 2

Next we compared the influence of emulsion size on its behavior. Intermediate density lipoproteins (IDL), very low density lipoproteins (VLDL) were combined as “Emulsion-S” and chylomicron sizes were marked as “Emulsion-L”. All emulsions were prepared as described in example 1. To produce larger emulsions (chylomicron-size), the neutral lipid/phospholipid ratio of the original mixture was increased to 4-5;1 and shorter sonication times were used (10-20 min). The size of the particles was measured using standard techniques.

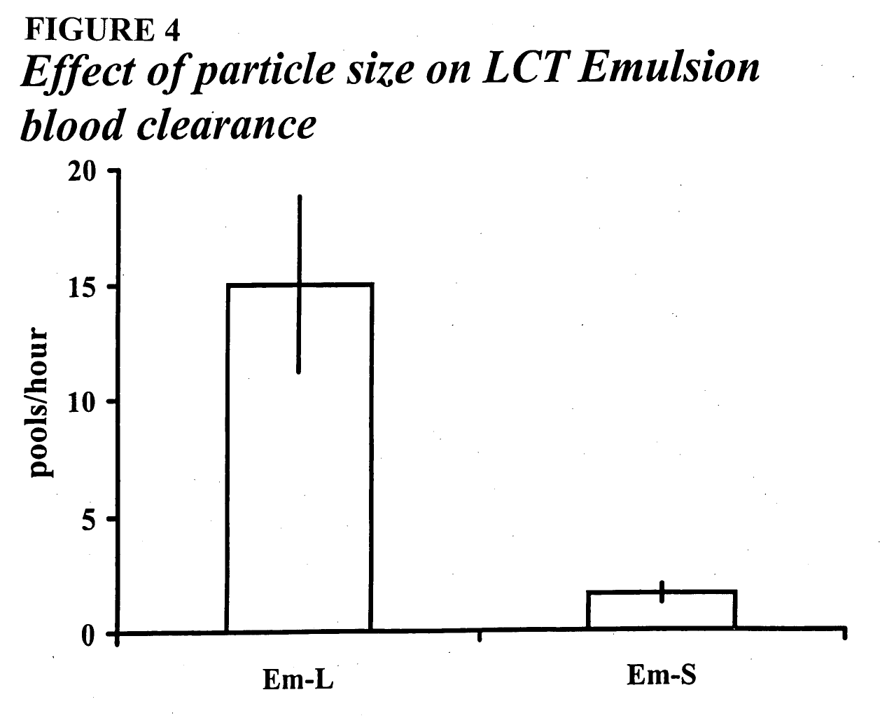

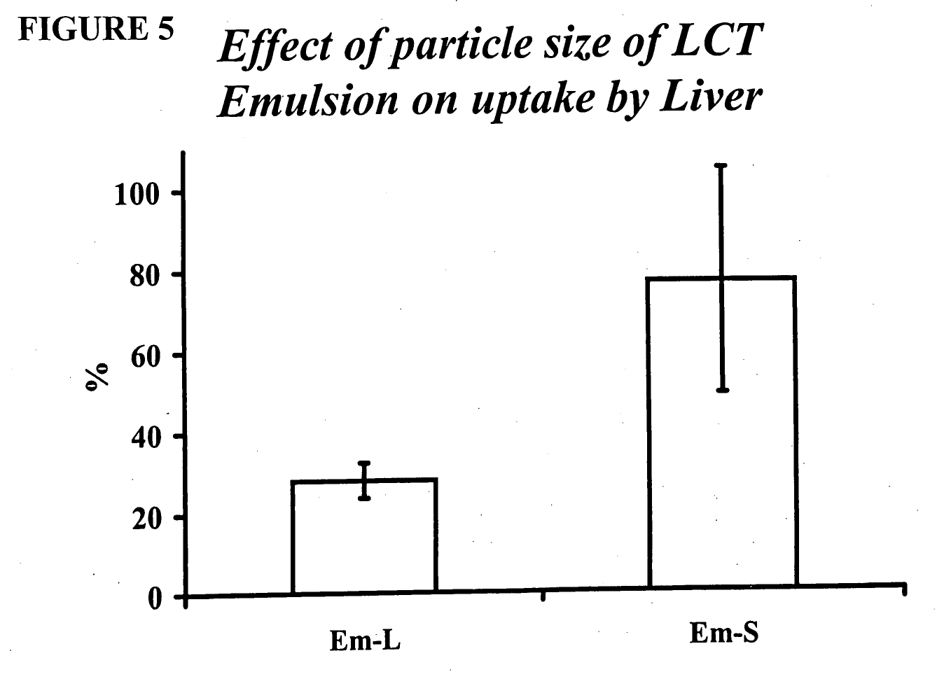

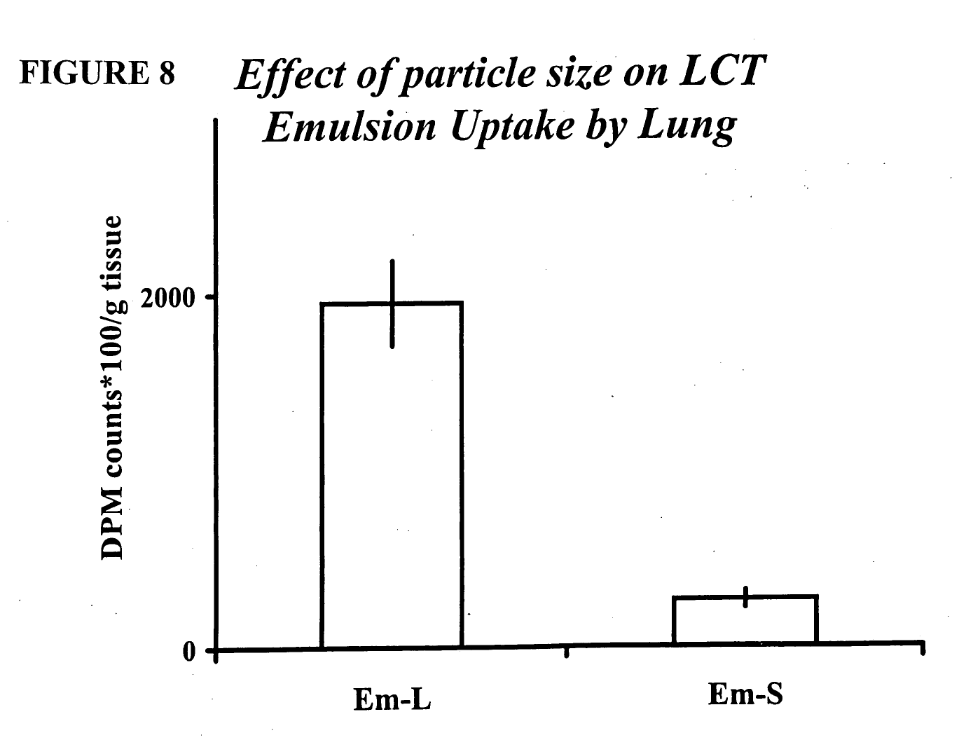

The blood clearance of IDL and VLDL (Emulsion-S) vs. chylomicron size particles (Emulsion-L) showed a 10 times faster clearance for the chylomicron type particles (1.2±0.3 pools/hr, 15±3.8 pools/hr, p<0.0001) (FIG. 4). Liver had 2 times higher uptake of VLDL vs. IDL size particles (56×10³±10×10³ DPM/gm vs. 28×10³±4×10³ DPM/gm). Percent wise Emulsion-S had significantly higher uptake than that of Emulsion-L (71%±3.1%, vs. 28%±4.3%, p<0.0001) (FIG. 5). For the lung, heart, spleen and kidney Emulsion-L uptake vs. Emulsion-S was significantly higher. For lungs it was 7.2 times higher (195×10³±24×10³ DPM/gm, vs. 27×10³±3×10³ DPM/gm, p<0.0001) (FIG. 8). For heart the difference between IDL, VLDL and chylomicron size emulsions was significant at 23 and 10 times respectively (531×10³±50×10³ DPM/gm, vs. 23×10³±2×10³ DPM/gm, 49×10³±7×10³ DPM/gm, p<0.0001). The spleen showed 19 and 16 times difference (700×10³±150×10³ DPM/gm, vs. 36×10³±2×10³ DPM/gm, 43×10³±7×10³ DPM/gm, p<0.0003). And kidney demonstrated 5.5 and 6.5 difference (91×10³±17×10³ DPM/gm, vs. 17×10³±2×10³ DPM/gm, 14×10³±3×10³ DPM/gm, p<0.0002).

### EXAMPLE 3

The LCT emulsion was produced as described in example 1. Incorporation of Apolipoprotein E or other ligands was performed by standard procedure. E. coli with DNA recombinant human ApoE3 was provided by Bio-technology General LTD, Rehovot, Israel.

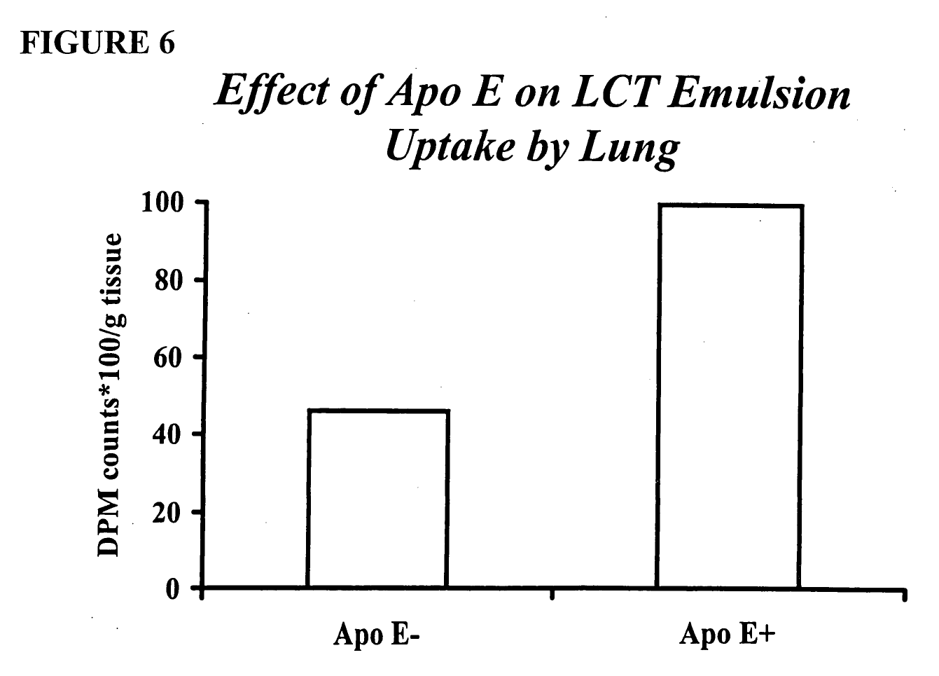

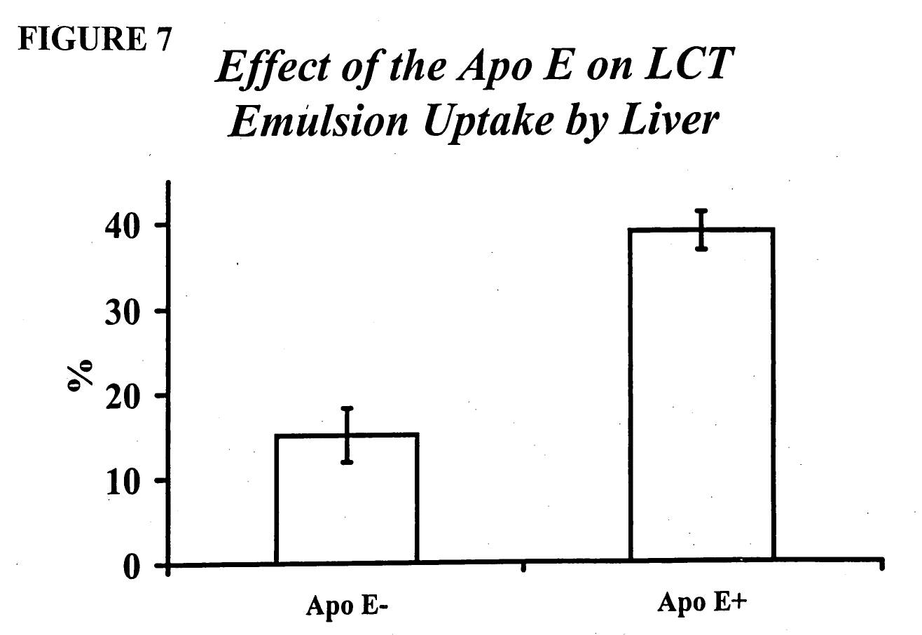

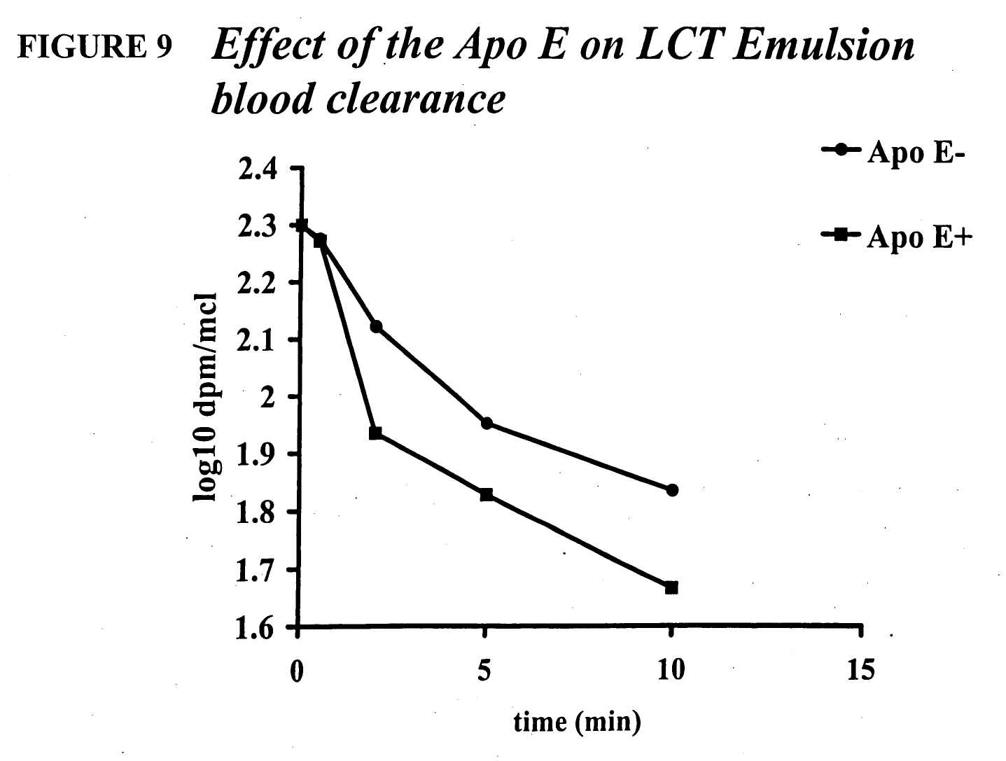

The addition of apolipoprotein E to the LCT emulsion increased the emulsion clearance (6.6±1.4 pools/hr, 7.2±0.4 pools/hr) (FIG. 9). The LCT emulsion, containing apolipoprotein E had higher liver uptake than the apolipoprotein E negative emulsion (39%±6%, vs. 15%±2%, p=0.01) (FIG. 7). There was also increase in lung uptake of the apolipoprotein E containing vs. apolipoprotein E negative emulsion (10×10³±1×10³ DPM/gm vs. 4.6×10³±0.3×10³ DPM/gm) (FIG. 6). Apolipoprotein E can help targeting, it binds tissues from liver to reticulo-endothelial, and binds to low density lipoprotein receptor, low density lipoprotein-related protein receptor, very low density lipoprotein receptor and cell surface proteglycans.

Methods and Materials

Emulsion preparation: Emulsions are prepared by standard industry methods for production of therapeutic emulsions in water. All emulsions were emulsified by egg yolk lecithin, 1.2 g/100 ml and contained 20 g Triglyceride/100 ml. The weight ratio of LCT, MCT, Ω-3 in the different composed triglyceride were varied according to choice. Standard desiccation, sonication, and ultracentrifugation procedures were subsequently performed as necessary. Emulsions were characterized by gel filtration and those emulsion and homogeneous fractions of constant size and lipid stoichiometry were pooled. Emulsions containing hydrophobic compounds or different surface or core lipids were prepared by incorporating such entities into the initial solvent mixture.

Preparation of different size emulsion particles: To produce larger emulsion particles (chylomicron-size), the neutral lipid/phospholipid ratio of the original mixture was increased to 4-5:1 and shorter sonication times were used (10-20 min).

Incorporation of Hydrophobic compounds: Hydrophobic compounds proposed for delivery were added to the emulsion, either during the original emulsion preparation or by sonication technique, to the existing emulsion. Elution profiles of emulsion on Sepharose CL2B column were used to show that all the. hydrophobic compound co-eluted with the emulsion particles.

Analysis of triglyceride and phospholipid levels: triglyceride levels are assayed by an enzymatic procedure using a commercial kit to the accompanying instructions (Boehringer Mannheim Diagnostics, Indianapolis, Ind.). Phospholipid levels were determined using the Bartlett procedure.

Animals: Pure bred C57BL/6J mice (Jackson Laboratory, Bar Harbor, Me.) were housed at room temperature at Columbia University animal facilities. They had access to standard pellet rodent chow (Laboratory Rodent Diet 5001, Richmond, Va.) and water ad libitum. For experiments, we used 8-16 week old mice, weighing 20-27 g each. Three sets of mice, 3-5 animals, for each of the 4 emulsions, were studied in each set of experiments. All experiments were initiated at 11:00 am. Anesthesia was provided by Avertin (Aldrich, Inc.) and injected intraperitoneally.

Animal Procedures: After anesthesia by intraperitoneal injection of Avertin, pain sensitivity was checked by tail or paw pinch. When the animal was unresponsive, with well preserved respiration, an incision was made in the inguinal area, and the femoral vein was visualized. Each emulsion was administered into the femoral vein as a bolus injection over 15 sec. Retro-orbital blood was collected at 0.5 min, 2 min, 5 min, 10 min, 15 min, and 25 min into capillary tubes at a volume of 20-75 μL. The length of the capillaries with blood was measured. The blood was kept at 40° C. before aliquoting for analyzes. Mice were sacrificed at 25 min, and the organs harvested. Organs sampled were liver, spleen, lungs, heart, soleus and gastrocnemius muscles, kidney, peritoneal fat, and brain. After rinsing the organs in the heparin solution 500 units/kg, tissues were weighed and stored at −200° C.

Liquid Scintillation Counting: Blood samples were transferred into 5 cc of Hydrofluor liquid scintillation counting solution (National Diagnostics, Atlanta, Ga.) and ³H-CE counts were assayed in Beckman LS 1800 Liquid Scintillation Counter. The tissues were homogenized using a Polytron Tissue Disrupter (Brinkmann Instruments, Westbury, N.Y.). Homogenates were extracted with volumes of chloroform-methanol (2:1 v/v) as described in. After vigorous mixing and centrifugation (to separate the two phases) at the speed of 2200 rotations/min, at 40° C. in the centrifuge TJ-6 (Beckman) and removal of the water phase, the chloroform solvent phase was evaporated. The resulting lipid phase was assayed in 20 mL of Hydrofluor liquid scintillation counting solution in a Beckman LS 1800 Liquid Scintillation Counter.

Calculations: Radioactivities are expressed per 1 L of blood. Fractional clearance rates are calculated based on 1st order linear kinetics observed during the first 10 min after injection. Total recovery of ³H-CE from all extracted tissues is calculated as 100%. ³H-CE counts in the liver are calculated as a percentage of total recovery. The hepatic vs. peripheral organ ³H-CE retention is expressed based on whole organ weight at the time of sacrifice. Results are presented as mean ±SE. Statistical analysis was carried out using one-way ANOVA.

Discussion

This work shows lipid particle property manipulation that allows the delivery of the carried biologically active substance in a predictable manner. The work shows a method for the preparation of a carrier with predictable delivery properties loaded with biologically active substance, where (1) lipid particle composition, (2) lipid particle size, (3) adjuvants for the lipid particle will determine and predict the speed of blood clearance and the identity of the tissue where the drug carried by the lipid particle is delivered to tissues.

1-30. (Canceled)

31. A method for preferentially delivering a pharmaceutical agent to a predefined tissue in a subject comprising administering to the subject a composition in the form of an emulsion comprising:
(a) a therapeutically effective amount of the pharmaceutical agent;
(b) an amount of a fish oil predetermined so as to deliver the pharmaceutical agent to the predefined tissue in the subject; and
(c) an amount of an emulsifier sufficient to result in the composition forming the emulsion,
so as to preferentially deliver the pharmaceutical agent to the predefined tissue in the subject.

32. The method of claim 31, wherein the fish oil is an Ω-3 triglyceride.

33. The method of claim 32, wherein the predefined tissue is an extrahepatic tissue and the Ω-3 triglyceride preferentially effects delivery of the pharmaceutical agent to the extrahepatic tissue.

34. A method for preferentially delivering a pharmaceutical agent to a predefined tissue in a subject comprising administering to the subject a composition in the form of an emulsion comprising:
(a) a therapeutically effective amount of the pharmaceutical agent;
(b) an amount of a medium chain triglyceride;
(c) an amount of a long-chain triglyceride; and
(d) an amount of an emulsifier sufficient to result in the composition forming the emulsion,
wherein the amount of the medium chain triglyceride relative to the amount of the long-chain triglyceride are predetermined so as to preferentially deliver the pharmaceutical agent to the predefined tissue in the subject.

35. The method of claim 34, wherein the amount of the medium chain triglyceride relative to the amount of the long-chain triglyceride is in a ratio of about one to one by weight.

36. A method for preferentially delivering a pharmaceutical agent to a predefined tissue in a subject comprising administering to the subject a composition in the form of an emulsion comprising:
(a) a therapeutically effective amount of the pharmaceutical agent;
(b) an amount of a fish oil;
(c) an amount of a medium chain triglyceride;
(d) an amount of a long-chain triglyceride; and
(e) an amount of an emulsifier sufficient to result in the composition forming an emulsion,
wherein each of the amount of fish oil, the amount of medium chain triglyceride and the amount of long-chain triglyceride is predetermined so as to preferentially deliver the pharmaceutical agent to the predefined tissue in the subject.

37. The method of claim 36, wherein the amount of the medium chain triglyceride relative to the amount of the long-chain triglyceride relative to the amount of the fish oil is in a ratio of about 5:4:1 by weight.

38. The method of claim 37, wherein the fish oil is an Ω-3 triglyceride.

39. The method of claim 38, wherein the predefined tissue is an extrahepatic tissue and the Ω-3 triglyceride preferentially effects delivery of the pharmaceutical agent to the extrahepatic tissue.

40. The method of any one of claims 31, 34, or 36, wherein the emulsion comprises particles and more than 80% of the particles in the emulsion have a diameter of between 30 and 150 nm.

41. The method of any one of claims 31, 34, or 36, wherein the emulsions comprises particles and more than 80% of the particles in the emulsion have a diameter of between 150 and 350 nm.

42. A method for delivering a pharmaceutical agent to a tissue expressing on its surface a low density lipoprotein receptor, a low density lipoprotein-related protein receptor, a very low density lipoprotein receptor or a proteoglycan in a subject comprising administering to the subject a composition in the form of an emulsion comprising:
(a) a therapeutically effective amount of the pharmaceutical agent;
(b) an amount of a triglyceride;
(c) an amount of an emulsifier sufficient to result in the composition forming the emulsion; and
(d) an amount of an apolipoprotein E which specifically binds to the predefined tissue,
wherein the amount of the triglyceride is predetermined to deliver the pharmaceutical agent to the tissue, and the amount of apolipoprotein E preferentially effects the delivery of the pharmaceutical agent to the tissue.

43. The method of claim 42, wherein the apolipoprotein E is human apolipoprotein E or a homolog thereof differing by fewer than 3 amino acids, but having the biological activity of naturally occurring human apolipoprotein E.

44. The method of claim 43, wherein the tissue is a hepatic tissue.

45. The method of claim 43, wherein the tissue is a reticulo-endothelial tissue.

46. The method of claim 31, 34, 36, or 42 wherein the administration comprises intravenous injection.
